(19) United States
(12) Patent Application Publication
(10) Pub. No.: US 2017/0260555 A1
(43) Pub. Date: Sep. 14, 2017

(54) METHOD FOR BIOSYNTHESIS OF ACETAMINOPHEN

(71) Applicant: The Regents of the University of California, Oakland, CA (US)

(72) Inventors: John Christopher Anderson, Berkeley, CA (US); Timothy Hsiau, Berkeley, CA (US); Saurabh Srivastava, Berkeley, CA (US); Paul Ruan, San Francisco, CA (US); Jonathan Prakash Inocencio Kotker, San Mateo, CA (US); Rastislav Bodik, Berkeley, CA (US); Sanjit A. Seshia, El Cerrito, CA (US)

(21) Appl. No.: 15/508,867

(22) PCT Filed: Sep. 25, 2015
(86) PCT No.: PCT/US15/52363

§ 371 Date: Mar. 3, 2017

Related U.S. Application Data
(60) Provisional application No. 62/056,866, filed on Sep. 29, 2014.

(51) Int. Cl. C12P 13/02; C12N 9/10; C12N 9/02

(52) U.S. Cl. CPC C12P 13/02; C12N 9/0073; C12N 9/1029; C12Y 114/13027; C12Y 203/01118

(57) ABSTRACT

The present disclosure provides methods for biosynthesis of acetaminophen. The present disclosure provides host cells genetically modified to provide for production of acetaminophen. The present disclosure provides a recombinant host cell that is genetically modified with one or more heterologous nucleic acids comprising nucleotide sequences encoding 4-aminobenzoate hydroxylase (4ABH) and N-hydroxyarylamine O-acetyltransferase (NhoA). The present disclosure provides a recombinant prokaryotic host cell that is genetically modified with one or more heterologous nucleic acids comprising nucleotide sequences encoding 44ABH and NhoA.

### CROSS-REFERENCE TO RELATED APPLICATIONS

Pursuant to 35 U.S.C. §119(e), this application claims the benefit of U.S. Provisional Patent Application Nos. 62/056,866, filed Sep. 29, 2014, which application is incorporated herein by reference in its entirety.

### STATEMENT REGARDING FEDERALLY SPONSORED RESEARCH

This invention was made with government support under Grant No. N66001-12-C-4204 awarded by the Defense Advanced Research Projects Agency. The government has certain rights in the invention.

### INCORPORATION BY REFERENCE OF SEQUENCE LISTING PROVIDED AS A TEXT FILE

A Sequence Listing is provided herewith as a text file, “BERK-261WO_SeqList_ST25.txt” created on Sep. 16, 2015 and having a size of 15 KB. The contents of the text file are incorporated by reference herein in their entirety.

### INTRODUCTION

N-acetyl-p-aminophenol, commonly known as acetaminophen, is a widely-used analgesic and antipyretic. Biosynthetic production of acetaminophen would provide a means for producing acetaminophen in vitro or in vivo.

### SUMMARY

The present disclosure provides host cells genetically modified to provide for production of acetaminophen. The present disclosure provides methods for biosynthesis of acetaminophen.

The present disclosure provides a recombinant host cell that is genetically modified with one or more heterologous nucleic acids comprising nucleotide sequences encoding 4-aminobenzoate hydroxylase (4ABH) and N-hydroxyarylamine O-acetyltransferase (NhoA). The present disclosure provides a recombinant prokaryotic host cell that is genetically modified with one or more heterologous nucleic acids comprising nucleotide sequences encoding 44ABH and NhoA. The present disclosure provides a recombinant eukaryotic host cell that is genetically modified with one or more heterologous nucleic acids comprising nucleotide sequences encoding 44ABH and NhoA. In some cases, the host cell comprises one or more endogenous nucleic acids comprises nucleotide sequences encoding polypeptides comprising amino acid sequences having at least 75%, at least 80%, at least 85%, at least 90%, at least 95%, at least 98%, or 100%, amino acid sequence identity to the amino acid sequences set forth in SEQ ID NOs:3, 4, and 5. In some cases, the host cell is genetically modified with one or more heterologous nucleic acids comprising nucleotide sequences encoding polypeptides comprising amino acid sequences having at least 75%, at least 80%, at least 85%, at least 90%, at least 95%, at least 98%, or 100%, amino acid sequence identity to the amino acid sequences set forth in SEQ ID NOs:3, 4, and 5. In some cases, the nucleotide sequences encoding polypeptides comprising amino acid sequences having at least 75%, at least 80%, at least 85%, at least 90%, at least 95%, at least 98%, or 100%, amino acid sequence identity to the amino acid sequences set forth in SEQ ID NOs:3, 4, and 5 are present on a single expression vector. In some cases, the nucleotide sequences encoding polypeptides comprising amino acid sequences having at least 75%, at least 80%, at least 85%, at least 90%, at least 95%, at least 98%, or 100%, amino acid sequence identity to the amino acid sequences set forth in SEQ ID NOs:3, 4, and 5 are operably linked to a promoter functional in the prokaryotic host cell. In some cases, the promoter is a constitutive promoter. In some cases, the expression vector is a medium copy expression vector. In some cases, the expression vector is a high copy expression vector. In some cases, the promoter is an inducible promoter. In some cases, the nucleotide sequences encoding 4ABH and the NhoA are present on a single expression vector, which may be a medium copy expression vector or a high copy expression vector. In some cases, the nucleotide sequences encoding 4ABH and the NhoA are integrated into the host cell's genome. In some cases, the nucleotide sequences encoding 4ABH and NhoA are operably linked to a constitutive promoter. In some cases, the nucleotide sequences encoding 4ABH and NhoA are operably linked to an inducible promoter. In some cases, the 4ABH comprises an amino acid sequence having at least 75%, at least 80%, at least 85%, at least 90%, at least 95%, at least 98%, or 100%, amino acid sequence identity to the amino acid sequence set forth in SEQ ID NO:1. In some cases, the NhoA comprises an amino acid sequence having at least 75%, at least 80%, at least 85%, at least 90%, at least 95%, at least 98%, or 100%, amino acid sequence identity to the amino acid sequence set forth in SEQ ID NO:2.

The present disclosure provides a method of producing N-acetyl-p-aminophenol in vitro, the method comprising culturing a recombinant prokaryotic host cell as described above, or elsewhere herein, in vitro in a culture medium and under conditions that provide for expression of the 4ABH and NhoA, wherein the cell produces p-aminobenzoic acid (PABA), wherein 4ABH catalyzes the conversion of PABA to produce p-aminophenol, and wherein the NhoA catalyzes the conversion of p-aminophenol to produce N-acetyl-p-aminophenol. In some cases, the method comprises purifying the N-acetyl-p-aminophenol produced by the host cell. In some cases, the host cell is Escherichia coli. In some cases, the host cell is a yeast cell, e.g., Saccharomyces cerevisiae. In some cases, the N-acetyl-p-aminophenol is produced in an amount of at least 50 mg/L culture medium.

The present disclosure provides a method of producing N-acetyl-p-aminophenol in vitro, the method comprising culturing the recombinant prokaryotic host cell as described above, or elsewhere herein, in vitro in a culture medium and under conditions that provide for expression of the 4ABH and NhoA, wherein the culture medium comprises p-aminobenzoic acid (PABA), wherein 4ABH catalyzes the conversion of PABA to produce p-aminophenol, and wherein the NhoA catalyzes the conversion of p-aminophenol to produce N-acetyl-p-aminophenol. In some cases, the method comprises purifying the N-acetyl-p-aminophenol produced by the host cell. In some cases, the host cell is a yeast cell, e.g., Saccharomyces cerevisiae. In some cases, the N-acetyl-p-aminophenol is produced in an amount of at least 50 mg/L culture medium.

The present disclosure provides a method of producing N-acetyl-p-aminophenol in an individual, the method comprising introducing into the individual the recombinant prokaryotic host cell as described above, or elsewhere herein, wherein the host cell produces N-acetyl-p-aminophenol in the individual.

The present disclosure provides a method of producing N-acetyl-p-aminophenol in an individual, the method comprising introducing into the individual one or more nucleic acids comprising nucleotide sequence encoding polypeptides comprising amino acid sequences having at least 75%, at least 80%, at least 85%, at least 90%, at least 95%, at least 98%, or 100%, amino acid sequence identity to the amino acid sequences set forth in SEQ ID NOs:1-5. In some cases, wherein the nucleic acids are present in a recombinant viral vector.

### BRIEF DESCRIPTION OF THE DRAWINGS

FIG. 1 depicts an engineered pathway to acetaminophen in Escherichia coli.

FIG. 2A-2E depict biosynthetic production of acetaminophen.

FIG. 3 depicts production of acetaminophen by 4AHB.NhoA cells when cultured in medium that includes p-aminobenzoic acid.

FIG. 4 provides an amino acid sequence of 4-aminobenzoate hydroxylase (4AHB).

FIG. 5 provides an amino acid sequence of N-hydroxyarylamine O-acetyltransferase (NhoA).

FIG. 6, FIG. 7, and FIG. 8 provide amino acid sequences of enzymes encoded by pabABC genes.

### DEFINITIONS

By “construct” or “recombinant vector” is meant a recombinant nucleic acid, generally recombinant DNA, which has been generated for the purpose of the expression of a specific nucleotide sequence(s), or is to be used in the construction of other recombinant nucleotide sequences.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “transformation” is used interchangeably herein with “genetic modification” and refers to a permanent or transient genetic change induced in a cell following introduction of new nucleic acid (i.e., DNA exogenous to the cell). Genetic change (“modification”) can be accomplished either by incorporation of the new DNA into the genome of the host cell, or by transient or stable maintenance of the new DNA as an episomal element. Where the cell is a eukaryotic cell, a permanent genetic change is generally achieved by introduction of the DNA into the genome of the cell. In prokaryotic cells, permanent changes can be introduced into the chromosome or via extrachromosomal elements such as plasmids and expression vectors, which may contain one or more selectable markers to aid in their maintenance in the recombinant host cell.

“Operably linked” refers to a juxtaposition wherein the components so described are in a relationship permitting them to function in their intended manner. For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

A “host cell,” as used herein, denotes an in vivo or in vitro eukaryotic cell, a prokaryotic cell, or a cell from a multicellular organism (e.g., a cell line) cultured as a unicellular entity, which eukaryotic or prokaryotic cells can be, or have been, used as recipients for a nucleic acid (e.g., an expression vector that comprises a nucleotide sequence encoding one or more biosynthetic pathway gene products such as mevalonate pathway gene products), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a subject prokaryotic host cell is a genetically modified prokaryotic host cell (e.g., a bacterium), by virtue of introduction into a suitable prokaryotic host cell a heterologous nucleic acid, e.g., an exogenous nucleic acid that is foreign to (not normally found in nature in) the prokaryotic host cell, or a recombinant nucleic acid that is not normally found in the prokaryotic host cell; and a subject eukaryotic host cell is a genetically modified eukaryotic host cell, by virtue of introduction into a suitable eukaryotic host cell a heterologous nucleic acid, e.g., an exogenous nucleic acid that is foreign to the eukaryotic host cell, or a recombinant nucleic acid that is not normally found in the eukaryotic host cell.

Expression cassettes may be prepared comprising a transcription initiation or transcriptional control region(s) (e.g., a promoter), the coding region for the protein of interest, and a transcriptional termination region. Transcriptional control regions include those that provide for over-expression of the protein of interest in the genetically modified host cell; those that provide for inducible expression, such that when an inducing agent is added to the culture medium, transcription of the coding region of the protein of interest is induced or increased to a higher level than prior to induction.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acids substitution groups are: valine-leucine-isoleucine, phenylalanine-tyrosine, lysine-arginine, alanine-valine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a genetically modified host cell” includes a plurality of such genetically modified host cells and reference to “the 4ABH gene” includes reference to one or more 4ABH genes and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides host cells genetically modified to provide for production of acetaminophen. The present disclosure provides methods for biosynthesis of acetaminophen.

### Recombinant Host Cells

The present disclosure provides a recombinant host cell that is genetically modified with one or more heterologous nucleic acids comprising nucleotide sequences encoding 4-aminobenzoate hydroxylase (4ABH) and N-hydroxyarylamine O-acetyltransferase (NhoA). In some cases, the host cell is a prokaryotic host cell. In some cases, the host cell is a eukaryotic host cell.

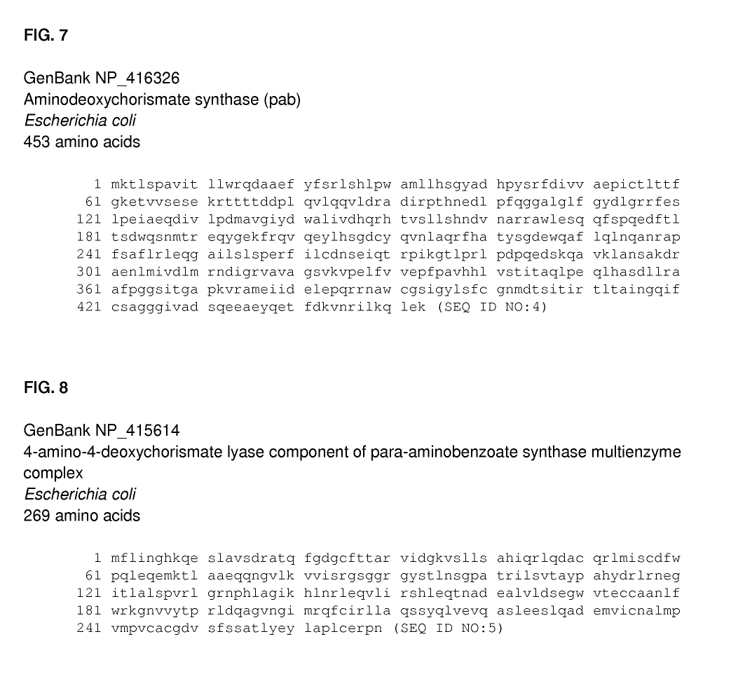

In some cases, a recombinant host cell of the present disclosure comprises endogenous pabABC genes that can encode polypeptides that provide for production of para-aminobenzoic acid (PABA) in the host cell. For example, in some cases, a recombinant host cell of the present disclosure comprises one or more endogenous nucleic acids comprising nucleotide sequences encoding polypeptides comprising amino acid sequences having at least 75%, at least 80%, at least 85%, at least 90%, at least 95%, at least 98%, or 100%, amino acid sequence identity to the amino acid sequences set forth in SEQ ID NOs:3, 4, and 5, and shown in FIG. 6, FIG. 7, and FIG. 8.

In some cases, a recombinant host cell of the present disclosure is genetically modified with one or more heterologous nucleic acids comprising nucleotide sequences encoding polypeptides comprising amino acid sequences having at least 75%, at least 80%, at least 85%, at least 90%, at least 95%, at least 98%, or 100%, amino acid sequence identity to the amino acid sequences set forth in SEQ ID NOs:3, 4, and 5. For example, in some cases, a recombinant host cell of the present disclosure is genetically modified with one or more heterologous nucleic acids comprising nucleotide sequences encoding: a) a polypeptide comprising an amino acid sequence having at least 75%, at least 80%, at least 85%, at least 90%, at least 95%, at least 98%, or 100%, amino acid sequence identity to the amino acid sequences set forth in SEQ ID NO:3; b) a polypeptide comprising an amino acid sequence having at least 75%, at least 80%, at least 85%, at least 90%, at least 95%, at least 98%, or 100%, amino acid sequence identity to the amino acid sequences set forth in SEQ ID NO:4; and c) a polypeptide comprising an amino acid sequence having at least 75%, at least 80%, at least 85%, at least 90%, at least 95%, at least 98%, or 100%, amino acid sequence identity to the amino acid sequences set forth in SEQ ID NO:5. In some cases, the nucleotide sequences encoding polypeptides comprising amino acid sequences having at least 75%, at least 80%, at least 85%, at least 90%, at least 95%, at least 98%, or 100%, amino acid sequence identity to the amino acid sequences set forth in SEQ ID NOs:3, 4, and 5 are present on a single expression vector. In some cases, the nucleotide sequences encoding polypeptides comprising amino acid sequences having at least 75%, at least 80%, at least 85%, at least 90%, at least 95%, at least 98%, or 100%, amino acid sequence identity to the amino acid sequences set forth in SEQ ID NOs:3, 4, and 5 are operably linked to a promoter functional in a prokaryotic host cell. In some cases, the nucleotide sequences encoding polypeptides comprising amino acid sequences having at least 75%, at least 80%, at least 85%, at least 90%, at least 95%, at least 98%, or 100%, amino acid sequence identity to the amino acid sequences set forth in SEQ ID NOs:3, 4, and 5 are operably linked to a promoter functional in a eukaryotic host cell.

In some cases, the nucleotide sequences encoding polypeptides comprising amino acid sequences having at least 75%, at least 80%, at least 85%, at least 90%, at least 95%, at least 98%, or 100%, amino acid sequence identity to the amino acid sequences set forth in SEQ ID NOs:3, 4, and 5 are operably linked to a promoter functional in a prokaryotic host cell, where the promoter is a constitutive promoter.

In some cases, the nucleotide sequences encoding polypeptides comprising amino acid sequences having at least 75%, at least 80%, at least 85%, at least 90%, at least 95%, at least 98%, or 100%, amino acid sequence identity to the amino acid sequences set forth in SEQ ID NOs:3, 4, and 5 are operably linked to a promoter functional in a prokaryotic host cell, where the promoter is an inducible promoter.

In some cases, the nucleotide sequences encoding polypeptides comprising amino acid sequences having at least 75%, at least 80%, at least 85%, at least 90%, at least 95%, at least 98%, or 100%, amino acid sequence identity to the amino acid sequences set forth in SEQ ID NOs:3, 4, and 5 are present on a single expression vector. In some cases, the expression vector is a medium copy expression vector. In some cases, the expression vector is a high copy expression vector.

In some cases, a recombinant host cell of the present disclosure is genetically modified with one or more nucleic acids comprising nucleotide sequences encoding 4ABH and NhoA, where the nucleotide sequences encoding the 4ABH and the NhoA are present on a single expression vector. In some cases, a recombinant host cell of the present disclosure is genetically modified with one or more nucleic acids comprising nucleotide sequences encoding 4ABH and NhoA, where the nucleotide sequences encoding the 4ABH and the NhoA are present on two different expression vectors. In some cases, the expression vector is a medium copy expression vector. In some cases, the expression vector is a high copy expression vector. In some cases, the expression vector is a low or single-copy expression vector. In some cases, the genes are expressed from the genome. In some cases, the nucleotide sequences encoding 4ABH and NhoA are operably linked to a constitutive promoter. In some cases, the nucleotide sequences encoding 4ABH and NhoA are operably linked to an inducible promoter. In some cases, the nucleotide sequence encoding 4ABH is operably linked to a constitutive promoter. In some cases, the nucleotide sequence encoding 4ABH is operably linked to an inducible promoter. In some cases, the nucleotide sequence encoding NhoA is operably linked to a constitutive promoter. In some cases, the nucleotide sequence encoding NhoA is operably linked to an inducible promoter. In some cases, the nucleotide sequences encoding 4ABH and NhoA are operably linked to separate promoters, while in other cases the two genes are expressed from a polycistronic operon.

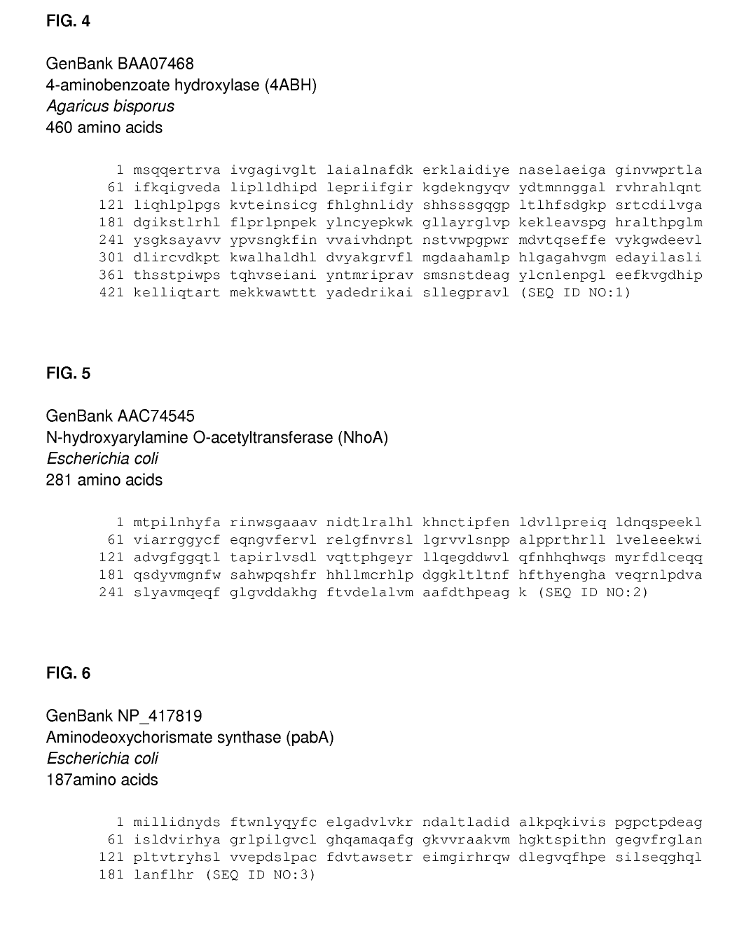

Suitable 4ABH polypeptides comprise an amino acid sequence having at least 75%, at least 80%, at least 85%, at least 90%, at least 95%, at least 98%, or 100%, amino acid sequence identity to the amino acid sequences set forth in SEQ ID NO:1 (FIG. 4).

Suitable NhoA polypeptides comprise an amino acid sequence having at least 75%, at least 80%, at least 85%, at least 90%, at least 95%, at least 98%, or 100%, amino acid sequence identity to the amino acid sequences set forth in SEQ ID NO:2 (FIG. 5).

As noted above, in some cases, a nucleotide sequence encoding one or more of a polypeptide having at least 75%, at least 80%, at least 85%, at least 90%, at least 95%, at least 98%, or 100%, amino acid sequence identity to an amino acid sequence set forth in one of SEQ ID NOs:1-5 is present in an expression vector. Suitable expression vectors include, but are not limited to, baculovirus vectors, bacteriophage vectors, transposons, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), conjugative recombinant DNAs (e.g. a Ti plasmid from Agrobacterium, or an RP4 plasmid from E. coli), P1-based artificial chromosomes, yeast plasmids, yeast artificial chromosomes, and any other vectors specific for specific hosts of interest (such as yeast). Thus, for example, a nucleic acid encoding a gene product(s) is included in any one of a variety of expression vectors for expressing the gene product(s). Such vectors include chromosomal, nonchromosomal and synthetic DNA sequences. The expression vector may also be a recombinant DNA inserted into the genome of the host cell using a genome engineering method (e.g. CRISPR/Cas9, multiplex automated genome engineering (MAGE), transposon mutagenesis, homing endonuclease-induced recombination, homologous recombination, or phage att site integration).

The nucleotide sequence in the expression vector is operably linked to an appropriate expression control sequence(s) (promoter) to direct synthesis of the encoded gene product. Depending on the host/vector system utilized, any of a number of suitable transcription and translation control elements, including constitutive and inducible promoters, transcription enhancer elements, transcription terminators, etc. may be used in the expression vector (see, e.g., Bitter et al. (1987) Methods in Enzymology, 153:516-544).

In addition, the expression vectors will in many embodiments contain one or more selectable marker genes (e.g., drug resistance) to provide a phenotypic trait for selection of genetically modified host cells.

As noted above, in some cases, a nucleotide sequence encoding one or more of a polypeptide having at least 75%, at least 80%, at least 85%, at least 90%, at least 95%, at least 98%, or 100%, amino acid sequence identity to an amino acid sequence set forth in one of SEQ ID NOs:1-5 is operably linked to an inducible promoter. Inducible promoters are well known in the art. Suitable inducible promoters include, but are not limited to, the pL of bacteriophage λ; Plac; Ptrp; Ptac (Ptrp-lac hybrid promoter); an isopropyl-beta-D-thiogalactopyranoside (IPTG)-inducible promoter, e.g., a lacZ promoter; a tetracycline-inducible promoter; an arabinose inducible promoter, e.g., PBAD (see, e.g., Guzman et al. (1995) J. Bacteriol. 177:4121-4130); a xylose-inducible promoter, e.g., Pxyl (see, e.g., Kim et al. (1996) Gene 181:71-76); a GAL1 promoter; a tryptophan promoter; a lac promoter; an alcohol-inducible promoter, e.g., a methanol-inducible promoter, an ethanol-inducible promoter; a raffinose-inducible promoter; a heat-inducible promoter, e.g., heat inducible lambda PL promoter, a promoter controlled by a heat-sensitive repressor (e.g., CI857-repressed lambda-based expression vectors; see, e.g., Hoffmann et al. (1999) FEMS Microbiol Lett. 177(2):327-34); and the like.

In yeast, a number of vectors containing constitutive or inducible promoters may be used. For a review see, Current Protocols in Molecular Biology, Vol. 2, 1988, Ed. Ausubel, et al., Greene Publish. Assoc. & Wiley Interscience, Ch. 13; Grant, et al., 1987, Expression and Secretion Vectors for Yeast, in Methods in Enzymology, Eds. Wu & Grossman, 31987, Acad. Press, N.Y., Vol. 153, pp.516-544; Glover, 1986, DNA Cloning, Vol. II, IRL Press, Wash., D.C., Ch. 3; Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II. A constitutive yeast promoter such as ADH or LEU2 or an inducible promoter such as GAL may be used (Cloning in Yeast, Ch. 3, R. Rothstein in: DNA Cloning Vol. 11, A Practical Approach, Ed. D M Glover, 1986, IRL Press, Wash., D.C.). Alternatively, vectors may be used which promote integration of foreign DNA sequences into the yeast chromosome.

In some cases, the host cell is a prokaryotic cell. Suitable prokaryotic cells include, but are not limited to, any of a variety of laboratory strains of Escherichia coli, Lactobacillus sp., Salmonella sp., Shigella sp., and the like. See, e.g., Carrier et al. (1992) J. Immunol. 148:1176-1181; U.S. Pat. No. 6,447,784; and Sizemore et al. (1995) Science 270:299-302. Examples of suitable Salmonella strains include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Bacillus subtilis, Pseudomonas pudita, Pseudomonas aeruginosa, Pseudomonas mevalonii, Rhodobacter sphaeroides, Rhodobacter capsulatus, Rhodospirillum rubrum, Rhodococcus sp., and the like. In some cases, the host cell is Escherichia coli.

In some cases, the host cell is a probiotic bacterium, e.g., Bifidobacterium, E. coli strain Nissle, Lactobacillus, and the like.

In some cases, the host cell is a eukaryotic cell. Suitable eukaryotic host cells include, but are not limited to, yeast cells, insect cells, plant cells, fungal cells, and algal cells. Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, Arabidopsis thaliana, Zea mays, Glycine max, Nicotiana tabacum, Cannabis indica, Beta vulgaris, Solanum, tuberosum, Solanum lycopersicum, Cucurbita pepo, Oryza sativa, and the like.

### Methods of Producing Acetaminophen in a Genetically Modified Host Cell In Vitro

The present disclosure provides a method of producing N-acetyl-p-aminophenol (acetaminophen) in a genetically modified host cell in vitro, the method comprising culturing a recombinant host cell of the present disclosure in vitro in a culture medium and under conditions that provide for expression of the 4ABH and NhoA, wherein the cell produces p-aminobenzoic acid (PABA), wherein 4ABH catalyzes the conversion of PABA to produce p-aminophenol, and wherein the NhoA catalyzes the conversion of p-aminophenol to produce N-acetyl-p-aminophenol. In some cases, the host cell is a prokaryotic host cell. In some cases, the host cell is Escherichia coli. In some cases, the host cell is a eukaryotic host cell. In some cases, the eukaryotic host cell is a yeast cell, e.g., Saccharomyces cerevisiae.

In some cases, a subject method for producing acetaminophen in a genetically modified host cell in vitro comprises culturing a genetically modified host cell in vitro in a culture medium comprising PABA, where the genetically modified host cell is genetically modified with one or more nucleic acids comprising nucleotide sequences encoding 4ABH and NhoA. The 4ABH catalyzes the conversion of PABA present in the host cell (where the PABA enters the cell from the culture medium) to produce p-aminophenol, and the NhoA catalyzes the conversion of p-aminophenol to produce N-acetyl-p-aminophenol. In some cases, the host cell is a prokaryotic host cell. In some cases, the host cell is Escherichia coli. In some cases, the host cell is a eukaryotic host cell. In some cases, the eukaryotic host cell is a yeast cell, e.g., Saccharomyces cerevisiae. The PABA present in the medium can be chemically synthesized (e.g., chemically synthesized in a cell-free system); can be purified from a source of PABA; or can be synthesized by another host cell present in the culture medium. In some case, PABA is present in the culture medium in a concentration of from about 1 mM to about 100 mM, e.g., from 1 mM to 10 mM, from 10 mM to 50 mM, from 50 mM to 75 mM, or from 75 mM to 100 mM.

A method of the present disclosure provides for production of N-acetyl-p-aminophenol in an amount of at least 0.5 mg/L, at least 1 mg/L, at least 2 mg/L, at least 5 mg/L, at least 10 mg/L, at least 15 mg/L, at least 20 mg/L. at least 25 mg/L, or more than 25 mg/L. A method of the present disclosure provides for production of N-acetyl-p-aminophenol in an amount of from 0.5 mg/L to 1 mg/L, from 1 mg/L to 5 mg/L, from 5 mg/L to 10 mg/L, from 10 mg/L to 15 mg/L, from 15 mg/L to 20 mg/L, from 20 mg/L to 25 mg/L, from 25 mg/L to 30 mg/L, from 30 to 50 mg/L, or more than 50 mg/L. In some cases, method of the present disclosure provides for production of N-acetyl-p-aminophenol in an amount of from 50 mg/L to 100 mg/L, from 100 mg/L to 250 mg/L, from 250 mg/L to 750 mg/L, from 750 mg/L to 1 g/L, or more than 1 g/L.

In some cases, a method of the present disclosure comprises purifying the N-acetyl-p-aminophenol produced by a host cell of the present disclosure. N-acetyl-p-aminophenol can be purified from culture medium, from cell lysate, or both. In some cases, the N-acetyl-p-aminophenol that is purified from the cell culture medium and/or cell lysate is at least 80% pure, at least 90% pure, at least 95% pure, at least 98% pure, at least 99% pure, or more than 99% pure, as assessed using any standard method such as liquid chromatograph-mass spectrometry and the like. The N-acetyl-p-aminophenol produced by a host cell of the present disclosure can be purified using solvent extraction, porous membranes, preparative chromatography, precipitation with salt, solvents, or polymers, solid-phase extraction, electrophoresis, crystallization, lyophilization, or a combination of these approaches.

### Methods of Producing Acetaminophen In Vivo or In Situ

The present disclosure provides a method of producing N-acetyl-p-aminophenol in an individual, the method comprising introducing into the individual a recombinant host cell of the present disclosure, wherein the host cell produces N-acetyl-p-aminophenol in the individual. In some cases, the individual is a human. In some cases, the individual is a non-human primate. In some cases, the individual is a non-human mammal such as a canine, a feline, a rodent (mouse; rat), a lagomorph (e.g., rabbit), a bovine, an equine, etc. In some cases, the recombinant host cell is a prokaryotic host cell, e.g., E. coli. In some cases, the recombinant host cell is a recombinant probiotic bacterial cell, and the recombinant cell is present in a probiotic formulation, a solid or semi-solid food product comprising the recombinant probiotic bacterial cell, or a liquid food product comprising the recombinant probiotic bacterial cell.

In some cases, a method of the present disclosure for producing N-acetyl-p-aminophenol in an individual comprises introducing into the individual one or more nucleic acids (e.g., a recombinant expression vector or genome engineering technology, or transiently transfected with DNAs or RNAs) comprising nucleotide sequence encoding one or more of: 1) a polypeptide having at least 75%, at least 80%, at least 85%, at least 90%, at least 95%, at least 98%, or 100%, amino acid sequence identity to the amino acid sequences set forth in SEQ ID NO:1; 2) a polypeptide having at least 75%, at least 80%, at least 85%, at least 90%, at least 95%, at least 98%, or 100%, amino acid sequence identity to the amino acid sequences set forth in SEQ ID NO:2; 3) a polypeptide having at least 75%, at least 80%, at least 85%, at least 90%, at least 95%, at least 98%, or 100%, amino acid sequence identity to the amino acid sequences set forth in SEQ ID NO:3; 4) a polypeptide having at least 75%, at least 80%, at least 85%, at least 90%, at least 95%, at least 98%, or 100%, amino acid sequence identity to the amino acid sequences set forth in SEQ ID NO:4; and 5) a polypeptide having at least 75%, at least 80%, at least 85%, at least 90%, at least 95%, at least 98%, or 100%, amino acid sequence identity to the amino acid sequences set forth in SEQ ID NO:5. In these cases, the host cell can be a prokaryotic host cell present in the individual (e.g., a prokaryotic host cell present in the intestine of the individual. In other cases, the host cell is a eukaryotic cell of the individual, e.g., an epithelial cell, an endothelial cell, and the like. In some cases, the one or more nucleic acids introduced into the individual is a recombinant expression vector.

In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, etc.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

In some cases, a recombinant host cell of the present disclosure is introduced into the lower gastrointestinal tract of the individual. In some cases, the recombinant host cell is introduced into the individual at or near a treatment site. In some cases, the recombinant host cell is encapsulated in a matrix; and the matrix is implanted into the individual. In some cases, the recombinant host cell is introduced into an individual orally. In some cases, the recombinant host cell is introduced into an individual rectally, e.g., by enema. In some cases, the recombinant host cell is introduced into an individual via a catheter.

In some cases, the individual has chronic pain. In some cases, the individual has chronic pain, and the recombinant host cell is introduced at or near a site of chronic pain. In some cases, the individual has inflammation. In some cases, the individual suffers from anxiety, depression, feelings of dread, or other psychological distress. In some cases, the individual suffers from chronic headaches. In some cases, the individual suffers from osteoarthritis. In some cases, the individual is unable to or has difficulty swallow pills. In some cases, the individual does not have access to a pharmacy due to physical isolation, and thus lacks access to purified acetaminophen, such as occurs during spaceflight or Antarctic travel.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Microbial Production of Acetaminophen

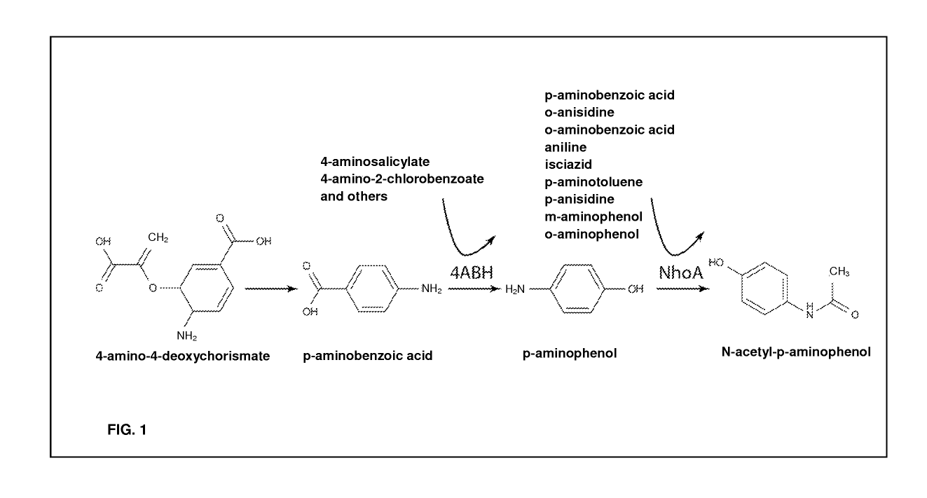

Using the Act retrosynthetic design algorithm (22), a potential biosynthetic route to acetaminophen was identified. This synthetic route is depicted schematically in FIG. 1. The predicted pathway branched off from chorismate biosynthesis, and was unnatural in two regards. First, it suggested a gene insertion from a mushroom (Agaricus bisporus) to convert p-aminobenzoic acid (PABA) to 4-aminophenol for the penultimate step. Second, it used an endogenous E. coli gene (nhoA) as the last step with an unnatural substrate. Although 4-aminophenol is not the natural substrate, the enzyme accepts it and acetylates it to acetaminophen.

FIG. 1. Engineered pathway to Acetaminophen in host E. coli. The target is predicted by Act to be three steps away from chorismate. The first gene taking 4-amino-4-deoxychorismate to p-aminobenzoic acid is native to E. coli. A single gene 4ABH from Agaricus bisporus was inserted to catalyze the conversion of p-aminobenzoic acid to p-aminophenol which is the substrate for a native E. coli gene nhoA whose product is acetaminophen. 4ABH has activity over substrates other than the one in this pathway, as shown, and so does NhoA. The substrates are shown in ascending order of Km values (23). NhoA's Km value for p-aminophenol is in between that of o-aminobenzoic acid and aniline.

The Agaricus bisporus gene 4ABH was synthesized in-house and the E. coli gene nhoA was cloned from genomic DNA. Genes were placed under the control of the constitutive promoter BBa_J23100 in a p15A plasmid (4ABH.NhoA, 4ABH, and NhoA constructs). The pabABC genes were assembled into an operon without a promoter and cloned into a high copy pUC vector (PabABC construct). As a biosafety precaution, a dapD knockout strain that strictly requires diaminopimelic acid for growth was employed (5). Acetaminophen-producing cells were generated via P1 transduction from the Keio collection (20) into the MC1061 derivative JTK165 (21), and then transformed with either the 4ABH.NhoA construct or both the PabABC and 4ABH.NhoA constructs.

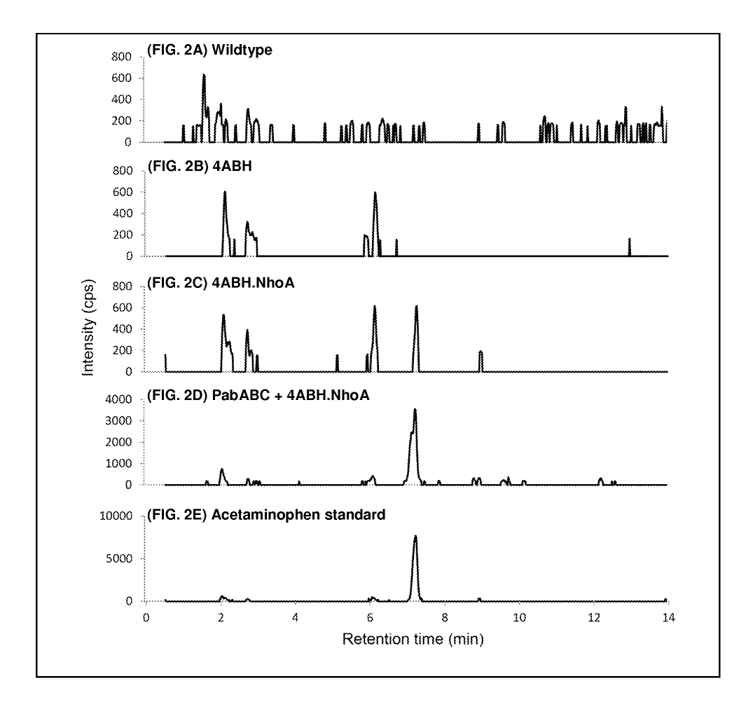

Liquid chromatograph-mass spectrometry (LC-MS) Analysis was performed on an Agilent 1260 Infinity HPLC and an Agilent 6120 Quadrupole LC-MS, using an Agilent Eclipse Plus C18 column. Transformants were grown in 4 mL cultures in glass test tubes with shaking for two days in GMML (Teknova) or one day in LB medium supplemented with antibiotics prior to analysis by LC-MS. Samples of fermentation broth with cells were desalted using Waters Oasis HLB Light Cartridges for both the experimental and background samples prior to injection. The “wildtype” background was the ΔdapD strain transformed with an empty vector. The positive control was a negative control with acetaminophen (Sigma Chemicals) doped into the fermentation broth prior to desalting. Titers were quantified against a standard curve. The results are shown in FIG. 2A-2E.

FIG. 2A-2E: Biosynthetic production of acetaminophen. Extracted positive ion chromatogram (m/z 152) from LC-MS analysis of cells containing (A) no added genes, (B) 4ABH, (C) 4ABH and NhoA, (D) 4ABH, NhoA, and PabABC, and (E) a standard of synthetically derived acetaminophen.

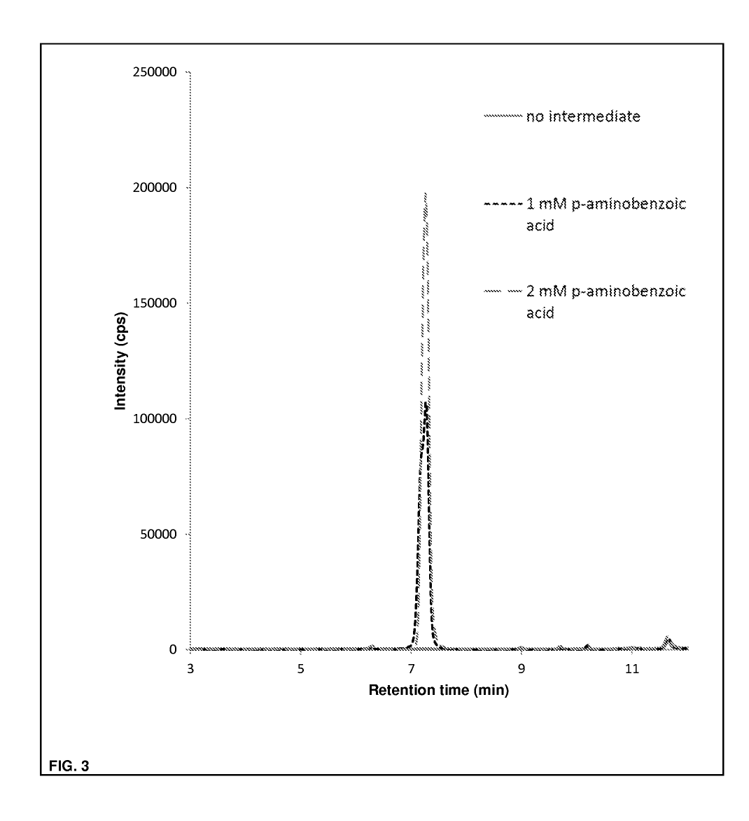

When 4ABH.NhoA cells are grown in minimal media, 1.8 μM acetaminophen was detected. The titer increased to 2.9 μM when PabABC was also included, suggesting that precursor availability is yield-limiting. When E. coli is grown in LB medium, the pabABC genes are repressed (24), and the acetaminophen pathway produces no product. This property allowed us to confirm the functions of the individual enzymes (4ABH and NhoA) using feeding experiments. When cells containing only NhoA were fed 10 mM p-aminobenzoic acid, 4-aminophenol was detected by observing colored oxidation products in the growth medium after an overnight culture. When cells containing 4ABH.NhoA were fed either precursor, acetaminophen was detected (FIG. 3).

FIG. 3: Precursor feeding. In LB media, 4ABH.NhoA cells only produce acetaminophen when fed p-aminobenzoic acid. Solid line is no intermediate added, short-dashed line (---) is 1 mM, and long-dashed line (--) is 2 mM.

### REFERENCES

1) D. K. Ro, et. al. Production of the antimalarial drug precursor artemisinic acid in engineered yeast. Nature. 440(7086), 940-943 (2006).
2) H. Yim. Metabolic engineering of Escherichia coli for direct production of 1,4-butanediol. Nat. Chem. Biol. 7(7), 445-452 (2011).
3) F. Zhu, et. al. In vitro reconstitution of mevalonate pathway and targeted engineering of farnesene overproduction in Escherichia coli. Biotechnol. Bioeng. in press (available at doi: 10.1002/bit.25198).
4) J. D. Keasling. Manufacturing molecules through metabolic engineering. Science. 330, 1355-1358 (2010).
5) G. H. Moe-Behrens, R. Davis, K. A. Haynes. Preparing synthetic biology for the world. Front Microbiol. 4, 5 (2013).
6) K. C. Soh, V. Hatzimanikatis. DREAMS of metabolism. Trends Biotechnol. 28(10), 501-508 (2010).
7) M. Kanehisa, S. Goto. KEGG: Kyoto Encyclopedia of Genes and Genomes. Nucleic Acids Res. 28,27-30 (2000)
8) J. Pramanik, J. D. Keasling, Stoichiometric model of Escherichia coli metabolism: incorporation of growth-rate dependent biomass composition and mechanistic energy requirements. Biotechnol. Bioeng.56(4), 398-421 (1997).
9) National Institutes of Health, NIH guidelines for research involving recombinant or synthetic nucleic acid molecules (NIH Guidelines) (2013, http://oba.od.nih.gov/oba/rac/Guidelines/NIH_Guidelines_prn.pdf)
10) Department of Health and Human Services, Screening Framework Guidance for Providers of Synthetic Double-Stranded DNA (2010, http://www.phe.gov/Preparedness/legal/guidance/syndna/Documents/syndna-guidance.pdf)
11) M. Liu, B. Bienfait, O. Sacher, J. Gasteiger, R. J. Siezen, A. Nauta, J. M. Geurts, Combining chemoinformatics with bioinformatics: in silico prediction of bacterial flavor-forming pathways by a chemical systems biology approach “reverse pathway engineering”. PLoS ONE.9(1), (2014).
12) E. J. Corey, A. K. Long, S. D. Rubenstein. Computer-assisted analysis in organic synthesis. Science 228, 408-418 (1985).
13) J. Law et al. Route Designer: a retrosynthetic analysis tool utilizing automated retrosynthetic rule generation. J. Chem. Inf. Model 49, 593-602 (2009).
14) W. D. Ihlenfeldt and J. Gasteiger. Computer-assisted planning of organic syntheses: The second generation of programs. Angewandte Chemie Intl. Ed. 34, 2613-2633 (1996).
15) M. Pförtner, M. Sitzmann. in Handbook of Chemoinformatics. J. Gasteiger, T. Engel, Eds. (Wiley, New York, 2003) pp. 1457-1507.
16) M. Sitzmann, M. Pförtner. in Handbook of Chemoinformatics. J. Gasteiger, T. Engel, Eds. (Wiley, New York, 2003) pp. 567-596.
17) M. Kanehisa, S. Goto, S. Kawashima, Y. Okuno, M. Hattori. The KEGG resource for deciphering the genome. Nucleic. Acids Res. 32, D277-280 (2004).
18) B. Ganesan, P. Dobrowolski, B. C. Weimer. Identification of the leucine-to-2-methylbutyric acid catabolic pathway of Lactococcus lactis. Appl. Environ, Microbiol. 72, 4264-4273 (2006).
19) C. H. Martin, D. R. Nielsen, K. V. Solomon, K. L. Prather. Synthetic metabolism: engineering biology at the protein and pathway scales. Chem. Biol. 16, 277-286 (2009).
20) T. Baba et. al. Construction of Escherichia coli K-12 in-frame, single-gene knockout mutants: the Keio collection. Mol. Syst. Biol. 2, 1-11 (2006).
21) J. T. Kittleson, S. Cheung, J. C. Anderson. Rapid optimization of gene dosage in E. coli using DIAL strains. J. Biol. Eng. 5, 5-10 (2011).
22) S. Srivastava, J. Kotker, S. Hamilton, P. Ruan, J. Tsui, J. C. Anderson, R. Bodik, S. A. Seshia. Pathway Synthesis using the Act Ontology, Fourth International Workshop on Bio-Design Automation (IWBDA) 2012, San Francisco, June 2012. http://www.eecs.berkeley.edu/˜sseshia/pubdir/iwbda12-act.pdf
23) E. Yamamura, M. Sayama, M. Kakikawa, M. Mori, A. Taketo, K. Kodaira. Purification and biochemical properties of an N-hydroxyarylamine O-acetyltransferase from Escherichia coli. Biochim. Biophys. Acta. 1475(1): 10-6 (2000).
24) J. Bongaerts, M. Kramer, U Muller, L. Raeven, M. Wubbolts. Metabolic engineering for microbial production of aromatic amino acids and derived compounds. Metab. Eng. 3(4), 289-300 (2001).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A recombinant prokaryotic host cell that is genetically modified with one or more heterologous nucleic acids comprising nucleotide sequences encoding 4-aminobenzoate hydroxylase (4ABH) and N-hydroxyarylamine O-acetyltransferase (NhoA).

2. The recombinant prokaryotic host cell of claim 1, wherein the host cell comprises one or more endogenous nucleic acids comprises nucleotide sequences encoding polypeptides comprising amino acid sequences having at least 75% amino acid sequence identity to the amino acid sequences set forth in SEQ ID NOs:3, 4, and 5.

3. The recombinant prokaryotic host cell of claim 1, wherein the host cell is genetically modified with one or more heterologous nucleic acids comprising nucleotide sequences encoding polypeptides comprising amino acid sequences having at least 75% amino acid sequence identity to the amino acid sequences set forth in SEQ ID NOs:3, 4, and 5.

4. The recombinant prokaryotic host cell of claim 3, wherein the nucleotide sequences encoding polypeptides comprising amino acid sequences having at least 75% amino acid sequence identity to the amino acid sequences set forth in SEQ ID NOs:3, 4, and 5 are present on a single expression vector.

5. The recombinant prokaryotic host cell of claim 3 or 4, wherein the nucleotide sequences encoding polypeptides comprising amino acid sequences having at least 75% amino acid sequence identity to the amino acid sequences set forth in SEQ ID NOs:3, 4, and 5 are operably linked to a promoter functional in the prokaryotic host cell.

6. The recombinant prokaryotic host cell of claim 5, wherein the promoter is a constitutive promoter.

7. The recombinant prokaryotic host cell of claim 4, wherein the expression vector is a medium copy expression vector.

8. The recombinant prokaryotic host cell of claim 4, wherein the expression vector is a high copy expression vector.

9. The recombinant prokaryotic host cell of claim 5, wherein the promoter is an inducible promoter.

10. The recombinant prokaryotic host cell of any one of claims 1-9, wherein the nucleotide sequences encoding 4ABH and the NhoA are present on a single expression vector.

11. The recombinant prokaryotic host cell of claim 10, wherein the expression vector is a medium copy expression vector.

12. The recombinant prokaryotic host cell of claim 10, wherein the expression vector is a high copy expression vector.

13. The recombinant prokaryotic host cell of any one of claims 1-12, wherein the nucleotide sequences encoding 4ABH and NhoA are operably linked to a constitutive promoter.

14. The recombinant prokaryotic host cell of any one of claims 1-12, wherein the nucleotide sequences encoding 4ABH and NhoA are operably linked to an inducible promoter.

15. The recombinant prokaryotic host cell of any one of claims 1-14, wherein the 4ABH comprises an amino acid sequence having at least 75% amino acid sequence identity to the amino acid sequence set forth in SEQ ID NO:1.

16. The recombinant prokaryotic host cell of any one of claims 1-14, wherein the 4ABH comprises an amino acid sequence having at least 85% amino acid sequence identity to the amino acid sequence set forth in SEQ ID NO:1.

17. The recombinant prokaryotic host cell of any one of claims 1-14, wherein the 4ABH comprises an amino acid sequence having at least 95% amino acid sequence identity to the amino acid sequence set forth in SEQ ID NO:1.

18. The recombinant prokaryotic host cell of any one of claims 1-14, wherein the NhoA comprises an amino acid sequence having at least 75% amino acid sequence identity to the amino acid sequence set forth in SEQ ID NO:2.

19. The recombinant prokaryotic host cell of any one of claims 1-14, wherein the NhoA comprises an amino acid sequence having at least 85% amino acid sequence identity to the amino acid sequence set forth in SEQ ID NO:2.

20. The recombinant prokaryotic host cell of any one of claims 1-14, wherein the NhoA comprises an amino acid sequence having at least 95% amino acid sequence identity to the amino acid sequence set forth in SEQ ID NO:2.

21. A method of producing N-acetyl-p-aminophenol in vitro, the method comprising culturing the recombinant prokaryotic host cell of any one of claims 1-20 in vitro in a culture medium and under conditions that provide for expression of the 4ABH and NhoA, wherein the cell produces p-aminobenzoic acid (PABA), wherein 4ABH catalyzes the conversion of PABA to produce p-aminophenol, and wherein the NhoA catalyzes the conversion of p-aminophenol to produce N-acetyl-p-aminophenol.

22. The method of claim 21, comprising purifying the N-acetyl-p-aminophenol produced by the host cell.

23. The method of claim 21, wherein the host cell is Escherichia coli.

24. The method of claim 21, wherein N-acetyl-p-aminophenol is produced in an amount of at least 50 mg/L culture medium.

25. A method of producing N-acetyl-p-aminophenol in vitro, the method comprising culturing the recombinant prokaryotic host cell of any one of claims 1-20 in vitro in a culture medium and under conditions that provide for expression of the 4ABH and NhoA, wherein the culture medium comprises p-aminobenzoic acid (PABA), wherein 4ABH catalyzes the conversion of PABA to produce p-aminophenol, and wherein the NhoA catalyzes the conversion of p-aminophenol to produce N-acetyl-p-aminophenol.

26. The method of claim 25, comprising purifying the N-acetyl-p-aminophenol produced by the host cell.

27. The method of claim 25, wherein the host cell is Escherichia coli.

28. The method of claim 25, wherein N-acetyl-p-aminophenol is produced in an amount of at least 50 mg/L culture medium.

29. A method of producing N-acetyl-p-aminophenol in an individual, the method comprising introducing into the individual the recombinant prokaryotic host cell of any one of claims 1-20, wherein the host cell produces N-acetyl-p-aminophenol in the individual.

30. A method of producing N-acetyl-p-aminophenol in an individual, the method comprising introducing into the individual one or more nucleic acids comprising nucleotide sequence encoding polypeptides comprising amino acid sequences having at least 75% amino acid sequence identity to the amino acid sequences set forth in SEQ ID NOs:1-5.

31. The method of claim 30, wherein the nucleic acids are present in a recombinant viral vector.


SEQUENCE LISTING (5 sequences)

SEQ ID NO: 1 - protein, 460 aa, Agaricus bisporus
MSQQERTRVAIVGAGIVGLTLAIALNAFDKERKLAIDIYENASELAEIGAGINVWPRTLAIFKQIGVEDALIPLLDHIPDLEPRIIFGIRKGDEKNGYQVYDTMNNGGALRVHRAHLQNTLIQHLPLPGSKVTEINSICGFHLGHNLIDYSHHSSSGQGPLTLHFSDGKPSRTCDILVGADGIKSTLRHLFLPRLPNPEKYLNCYEPKWKGLLAYRGLVPKEKLEAVSPGHRALTHPGLMYSGKSAYAVVYPVSNGKFINVVAIVHDNPTNSTVWPGPWRMDVTQSEFFEVYKGWDEEVLDLIRCVDKPTKWALHALDHLDVYAKGRVFLMGDAAHAMLPHLGAGAHVGMEDAYILASLITHSSTPIWPSTQHVSEIANIYNTMRIPRAVSMSNSTDEAGYLCNLENPGLEEFKVGDHIPKELLIQTARTMEKKWAWTTTYADEDRIKAISLLEGPRAVL

SEQ ID NO: 2 - protein, 281 aa, Escherichia coli
MTPILNHYFARINWSGAAAVNIDTLRALHLKHNCTIPFENLDVLLPREIQLDNQSPEEKLVIARRGGYCFEQNGVFERVLRELGFNVRSLLGRVVLSNPPALPPRTHRLLLVELEEEKWIADVGFGGQTLTAPIRLVSDLVQTTPHGEYRLLQEGDDWVLQFNHHQHWQSMYRFDLCEQQQSDYVMGNFWSAHWPQSHFRHHLLMCRHLPDGGKLTLTNFHFTHYENGHAVEQRNLPDVASLYAVMQEQFGLGVDDAKHGFTVDELALVMAAFDTHPEAGK

SEQ ID NO: 3 - protein, 187 aa, Escherichia coli
MILLIDNYDSFTWNLYQYFCELGADVLVKRNDALTLADIDALKPQKIVISPGPCTPDEAGISLDVIRHYAGRLPILGVCLGHQAMAQAFGGKVVRAAKVMHGKTSPITHNGEGVFRGLANPLTVTRYHSLVVEPDSLPACFDVTAWSETREIMGIRHRQWDLEGVQFHPESILSEQGHQLLANFLHR

SEQ ID NO: 4 - protein, 453 aa, Escherichia coli
MKTLSPAVITLLWRQDAAEFYFSRLSHLPWAMLLHSGYADHPYSRFDIVVAEPICTLTTFGKETVVSESEKRTTTTDDPLQVLQQVLDRADIRPTHNEDLPFQGGALGLFGYDLGRRFESLPEIAEQDIVLPDMAVGIYDWALIVDHQRHTVSLLSHNDVNARRAWLESQQFSPQEDFTLTSDWQSNMTREQYGEKFRQVQEYLHSGDCYQVNLAQRFHATYSGDEWQAFLQLNQANRAPFSAFLRLEQGAILSLSPERFILCDNSEIQTRPIKGTLPRLPDPQEDSKQAVKLANSAKDRAENLMIVDLMRNDIGRVAVAGSVKVPELFVVEPFPAVHHLVSTITAQLPEQLHASDLLRAAFPGGSITGAPKVRAMEIIDELEPQRRNAWCGSIGYLSFCGNMDTSITIRTLTAINGQIFCSAGGGIVADSQEEAEYQETFDKVNRILKQLEK

SEQ ID NO: 5 - protein, 270 aa, Escherichia coli
PMFLINGHKQESLAVSDRATQFGDGCFTTARVIDGKVSLLSAHIQRLQDACQRLMISCDFWPQLEQEMKTLAAEQQNGVLKVVISRGSGGRGYSTLNSGPATRILSVTAYPAHYDRLRNEGITLALSPVRLGRNPHLAGIKHLNRLEQVLIRSHLEQTNADEALVLDSEGWVTECCAANLFWRKGNVVYTPRLDQAGVNGIMRQFCIRLLAQSSYQLVEVQASLEESLQADEMVICNALMPVMPVCACGDVSFSSATLYEYLAPLCERPN